(19) United States
(12) Patent Application Publication
(10) Pub. No.: US 2003/0212325 A1
(43) Pub. Date: Nov. 13, 2003

(54) Method for determining a dose distribution in radiation therapy

(76) Inventors: Cristian Cotrutz, Redwood City, CA (US); Lei Xing, Stanford, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 10/388,201

(22) Filed: Mar. 12, 2003

Related U.S. Application Data
(60) Provisional application No. 60/363,913, filed on Mar. 12, 2002.

(51) Int. Cl. A61B 6/00

(52) U.S. Cl. 600/436

(57) ABSTRACT

A method is provided for interactive treatment planning of IMRT or other radiation modalities that employs non-uniform tuning or optimization. One aspect provides a voxel-dependent penalty scheme by varying the importance factor associated with a voxel, the prescription at the voxel, or the form of the penalty function at the voxel in a non-uniform manner. Another aspect provides the dose shape at a specified sub-volume tuned by varying the local importance factor(s) or the local prescription or the form/value of penalty function. Yet another aspect provides the use of a non-uniform penalty scheme (non-uniform importance factors, non-uniform prescription in one or more structures, or non-uniform form of the objective function). Still another aspect provides the method of pre-estimating the values of the voxel-specific importance factors using prior dosimetric knowledge of a given system.

### CROSS-REFERENCE TO RELATED APPLICATIONS

This application is cross-referenced to and claims priority from U.S. Provisional application 60/363,913 filed Mar. 12, 2002, which is hereby incorporated by reference.

### STATEMENT REGARDING FEDERALLY SPONSORED RESEARCH OR DEVELOPMENT

This invention was supported in part by grant number Army DAMD 17-01-1-0635 from the U.S. Department of Defense. The U.S. Government has certain rights in the invention.

### FIELD OF THE INVENTION

The present invention relates generally to radiation therapy. More particularly, the present invention relates to a method for determining a dose distribution in intensity modulated radiation therapy with non-uniform parameters that affect local dosimetric behavior.

### BACKGROUND

Inverse modulated radiation therapy (IMRT) represents one of the most important advancements in radiation therapy. IMRT aims at delivering high radiation doses to target volumes while minimizing radiation exposure of adjacent critical structures. IMRT inverse planning is usually performed by pre-selecting parameters like beam modality, beam configuration and importance factors and then optimizing the fluence profiles or beamlet weights. The beam profiles of an IMRT treatment are usually obtained using inverse planning. Examples of such approaches can be found in, for instance, Webb (1989) in a paper entitled “Optimisation of conformal radiotherapy dose distributions by simulated annealing” and published in “Phys. Med. Biol. 34(10):1349-70”; Bortfeld et al. (1990) in a paper entitled “Methods of image reconstruction from projections applied to conformation radiotherapy” and published in “Phys. Med. Biol. 35(10):1423-34”; Xing et al. (1996) in a paper entitled “Iterative algorithms for Inverse treatment planning” and published in “Phys. Med. Biol. 41(2):2107-23”; Olivera et al. (1998) in a paper entitled “Maximum likelihood as a common computational framework in tomotherapy” and published in “Phys. Med. Biol. 43(11):3277-94”; Spirou et al. (1998) in a paper entitled “A gradient inverse planning algorithm with dose-volume constraints” and published in “Med. Phys. 25(3):321-33”; in Wu et al. (2000) in a paper entitled “Algorithms and functionality of an intensity modulated radiotherapy optimization system” and published in “Med. Phys. 27(4):701-11”; and Cotrutz et al (2001) in a paper entitled “A multiobjective gradient-based dose optimization algorithm for external beam conformal radiotherapy” and published in “Phys. Med. Biol. 46(8) 2161-2175”.

The approach of minimization of an objective function with dose-volume constraints attempts to satisfy the dose-volume constraints either by constantly penalizing those voxels that exceed the permitted fractional volume (See Spirou et al. 1998 which is the same paper as referenced supra) or by adopting a volume sensitive variable penalization scheme (See Cho et al. 1998 which is the same paper as referenced supra) of the same voxels. The final solution is determined by the choice of DVH prescriptions and the structure specific importance factors that prioritize the relative importance of the clinical goals of the involved structures. In reality, the IMRT dose optimization problem may be ill-conditioned and there may not be a solution to account for the chosen parameters and constraints. A planner is often required to conduct a multiple trial-and-error process where several parameters are sequentially tried until an acceptable compromise is achieved. The resulting solution reflects a balance between the conflicting requirements of the target and the sensitive structures. A problem of the conventional inverse planning formalism is that there exists no effective mechanism for a planner to fine-tune the dose distribution at a local level or to differentially modify the dose-volume histograms (DVHs) of the involved structures. Accordingly there is a need in the art to develop new methods to determine the dose distributions at a local level to overcome the shortcomings in the current methods.

### SUMMARY OF THE INVENTION

The present invention provides an effective mechanism for interactive treatment planning of IMRT or other radiation modalities employing non-uniform tuning or optimization. In a first aspect of the invention, a treatment plan is determined using two steps. The first step is based on conventional inverse planning, where the structure specific importance factors are determined and the corresponding beam parameters (e.g. beam profiles) are optimized under the guidance of a conventional objective function. In the second step, the “optimal” plan is then fine-tuned by modifying the voxel dependent importance factor to meet a clinical requirement. For every change in the regional importance factors, the beam parameters need to be re-optimized. This process continues in an iterative fashion until a satisfactory solution is obtained. In another aspect of the invention the non-uniform parameters to change to local dosimetric behavior could also be used in the first step.

In another aspect of the invention, a method with a voxel-dependent penalty scheme for inverse treatment planning is provided. The voxel-dependent penalty scheme is realized by varying the importance factor associated with a voxel, the prescription at the voxel, or the form of the penalty function at the voxel in a non-uniform manner. This way the penalty will not only depend on the dose discrepancy at a voxel but also the importance factor or the local penalty value.

In yet another aspect of the invention, a method is provided to effectively fine-tune the dose shape at a specified sub-volume by varying the local importance factor(s) or the local prescription or the form/value of penalty function. The voxel-dependent penalty scheme provides a valuable mechanism for interactive planning of IMRT treatment. In addition to adjust the local importance factors directly (e.g., graphically pointing out the region(s) where the dose need to be changed), the voxel dependent importance factors can also be adjusted implicitly through the guidance of the DVH curves of different structures. In this case, one may point out (graphically) which part of a DVH curve should be changed and toward which direction, the system will find the corresponding voxels in the structure and adjust their importance factors accordingly. It is noted that the system is a correlated system and for every adjustment of the local importance factor(s), the beam profiles need to be re-optimized and the final solution needs to be re-evaluated. The approach provides one with control over the spatial dose distribution.

In still another aspect of the invention the use of non-uniform penalty scheme (non-uniform importance factors, non-uniform prescription in one or more structures, or non-uniform form of the objective function) and/or the method of pre-estimating the values of the voxel-specific importance factors using prior dosimetric knowledge of the given system is provided. For a given beam configuration and a given patient with pre-specified target dose prescription and the tolerance doses of the sensitive structures, the dosimetric capability at a target voxel is measured by the “distance” between the prescribed target dose and the best achievable dose without violating the dosimetric constraints of the sensitive structures. For a voxel in a sensitive structure, the capability evaluation is similar except that one may now require the system to meet the target prescription first and then examine the dose in the sensitive structures relative to their tolerances. The capabilities of the voxels contain a priori geometric and dosimetric information of the system. The voxel-based inverse planning is more efficient by taking into account the dosimetric capability of the involved voxels when one adjusts the local importance factors.

An advantage of the present invention is that the voxel-dependent importance factors and/or penalty scheme greatly enlarge the IMRT plan solution space and makes it possible to find better solutions compared to current methods. Furthermore, the voxel-dependent penalty scheme (e.g., voxel-dependent importance factors, or voxel-dependent prescription, or voxel-dependent penalty function) can be used as a means for interactive IMRT planning. This provides a direct way to fine-tune the dose distribution through the adjustment of responding local penalty parameter. Yet another advantage is that the present invention provides a method of pre-estimation of voxel-dependent importance factors based on the voxel-dependent dosimetric capability and the method of computing the dosimetric capability at a given voxel. This allows one to incorporate a priori system knowledge into the planning process and speeds up the determination of local importance factors.

### BRIEF DESCRIPTION OF THE FIGURES

The objectives and advantages of the present invention will be understood by reading the following summary in conjunction with the drawings, in which:

FIG. 1 shows an embodiment of an interactive inverse planning method with voxel-dependent importance factors according to the present invention;

FIG. 2 shows an example of a C-shaped tumor and a nine-beam setup used for dose optimization. Dose prescription is set 100 dose units (arbitrary units) to the tumor (PTV) and 20 units to the circular critical structure (CSV);

FIG. 3 shows an example of dose volume histograms (DVHs) corresponding to three optimization runs, with different values of the local importance factors. Dose is normalized to the mean target dose;

FIG. 4 shows an example of a transversal slice showing the anatomical structures delineated for the nasopharinx tumor and the corresponding optimized dose distribution for local importance factors of unit value. The doses are normalized to the mean target value;

FIG. 5 shows an example of DVHs for plans optimized with unit value local importance factors (the plain lines) versus plans optimized using higher value of local importance factors for the right eye (the dashed lines);

FIG. 6 shows an example of DVHs for plans optimized with unit value local importance factors (the plain lines) versus plans optimized using higher value of local importance factors for both the eye structures (the dashed lines);

FIG. 7 shows an example of DVHs for plans optimized with unit value local importance factors (the plain lines) versus plans optimized using higher value of local importance factors for both the eye structures and the optic chiasm (the dashed lines);

FIG. 8 shows an example of DVHs of three prostate plans: (a) Prostate; (b) Bladder; (c) Rectum; (d) Right Femoral head; (e) Left Femoral Head. The gray lines represent the conventionally optimized plan. The black solid and dotted lines correspond to plans optimized with voxel importance factors of 2 and 3, respectively. These values were assigned for those voxels accounted within the 80-88% dose interval (vertical lines in FIG. 8A);

FIG. 9 shows an example of a isodose plot showing the 85% isodose lines corresponding to the three prostate optimizations. The inner isodose corresponds to the conventional optimization (rₙ=1) and the outer lines to the optimizations performed with values of the regional importance factors of rₙ=2 and rₙ=3, respectively;

FIG. 10 shows an example of a dose distribution for a conventional optimized prostate IMRT plan. Two tumor hot spots of 106% are present within the prostate;

FIG. 11 shows an example of a prostate dose distribution after dose shaping by increasing the regional importance factors. The left 106% tumor hot spot in FIG. 10 disappeared completely while the second has reduced its size considerably; and

FIG. 12 shows an example of DVHs of (a) Prostate; (b) Bladder; (c) Rectum; (d) Right Femoral head; (e) Left Femoral Head for three IMRT plans. The gray lines represent the conventionally plan, the black solid and dotted lines correspond to plans optimized with voxel importance factors of 2 and 3, respectively. These values were assigned for those voxels accounted within the 105-110% dose interval (vertical lines in FIG. 12a).

### DETAILED DESCRIPTION OF THE INVENTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the present invention is set forth without any loss of generality to, and without imposing limitations upon, the claimed invention.

The problem in inverse radiotherapy is to determine a vector of beamlet weights, w, to achieve a prescribed dose distribution or DVHs. In vector form, the dose to the points in the treatment region or target volume depends upon the beamlet weights as:

D_{c}=d·w (1)

where d represents the dose deposition coefficient matrix, expressing the dose deposited to any calculation point when irradiated with a set of unit weight beamlets. A method to minimize the problem in inverse radiotherapy is to use a quadratic objective function defined by:

$$F = \frac{1}{N} {\sum }_{n} {r}_{σ} \cdot {[ {D}_{c} ( n ) - {D}_{0} ( n ) ]}^{2} \qquad ( 2 )$$

where D_{c} and D₀ are the calculated and prescribed doses respectively, N is the total number of voxels within a target volume or structure σ, n is the voxel index, and r_{σ} is the importance factor that controls the relative importance of a structure a (See, for instance, Webb (1989) in a paper entitled “Optimisation of conformal radiotherapy dose distributions by simulated annealing” and published in “Phys. Med. Biol. 34(10):1349-70”; Bortfeld et al. (1990) in a paper entitled “Methods of image reconstruction from projections applied to conformation radiotherapy” and published in “Phys. Med. Biol. 35(10):1423-34”; or Xing et al. (1996) in a paper entitled “Iterative algorithms for Inverse treatment planning” and published in “Phys. Med. Biol. 41(2):2107-23”). Different sets of importance factors result in different “optimal” solutions and multiple trial-and-error are often needed to find a set of clinically acceptable values. Several computer methods have been proposed to facilitate the trial-and-error determination of the importance factors (See, for instance, Xing et al. (1999) in a paper entitled “Optimization of importance factors in inverse planning” and published in “Phys. Med. Biol. 44(10):2525-36”; Xing et al. (1999) in a paper entitled “Estimation theory and model parameter selection for therapeutic treatment plan optimization” and published in “Med. Phys. 26(11):2348-58”; Cotrutz et al. (2001) in a paper entitled “A multiobjective gradient-based dose optimization algorithm for external beam conformal radiotherapy” and published in “Phys. Med. Biol. 46(8):2161-2175”; and Wu et al. (2001) in a paper entitled “An optimization method for importance factors and beam weights based genetic algorithms for radiotherapy treatment planning” and published in “Phys. Med. Biol. 46 1085-99”).

One aspect of the present invention is a general inverse-planning framework with non-uniform importance factors. In this new formalism, the importance at a voxel n is expressed as a product of two factors, r_{σ} and rₙ (see Eq. 3), where r_{σ} characterizes the importance of the structure σ (target volume) as an entity relative to other structures (sub-volumes), and rₙ modulates the importance in obtaining an optimal solution at a regional (sub-volume) level of the structure (target volume). The voxel-specific importance factor provides an effective means to prioritize the inner-structural importance. The objective function now reads:

$$F = {\sum }_{σ = 1}^{{n}_{σ}} ⌊ \frac{1}{{N}_{σ}} {\sum }_{n = 1}^{{N}_{σ}} {r}_{σ} \cdot {r}_{n} \cdot {[ {D}_{c} ( n ) - {D}_{0} ( n ) ]}^{2} ⌋ \qquad ( 3 )$$

where N_{σ} represents the total number of voxels of a structure. In Eq 3, D₀(n) is the prescription dose. Note that conventional inverse planning scheme represents a special case of the more general formalism proposed here when all the rₙ's have unit values.

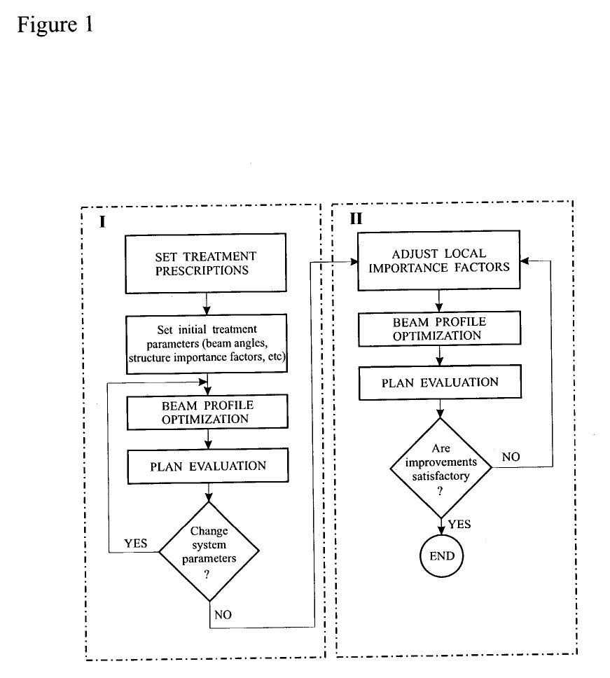

FIG. 1 shows another aspect of the present invention with an overall planning method for dose optimization. The overall planning includes two main steps. The first main step as shown by rectangle I, represents the conventional inverse planning process, where system parameters, such as structure-specific importance factors and beam angles, are determined through trial-and-error. For each trial, the optimization results are assessed using dose distributions and DVH tools, which can be realized by any inverse planning system common in the art. After the conventional IMRT plan is obtained, the method as shown in FIG. 1 proceeds to the next stage of interactive planning shown in rectangle II. The flow of method steps in rectangle II follows a similar pattern as in the case of conventional planning (rectangle I), however with the main difference that now the adjustment of parameters are performed to the local importance factors in a non-uniform manner. The local importance factors are also referred to in this invention as sub-volume dependent parameters, i.e. for instance voxel-based or penalty function based parameters. The method in rectangle II is iterative, wherein every cycle of this iterative procedure begins with the assessment of the dose distributions and DVHs resulted from the precedent loop, i.e. either the end result from rectangle I or when local importance factors are included and the dose distributions have been re-optimized in rectangle II. The fine-tuning can also be done based on the evaluation of the DVH curve(s). The planner selects the dose interval(s) for which further refinement of structure DVH(s) is(are) sought. The indices of the voxels belonging to the selected dose interval(s) are detected and “turned on”. The local importance factors of these voxels are then increased or decreased accordingly. Increasing the values of the local importance factors will increase the penalty level at the considered voxels and generally will lead to a better compliance of the resulting dose distribution with the prescription in that region or sub-volume. Decreasing the importance factors will have an opposite effect and relax the compliance of the resulting dose distribution with the prescription in that region or sub-volume. In one embodiment, the amount of change in the importance factors could be established empirically. In another embodiment, the amount of change in the importance factors could be determined by assigning a value, e.g. 15˜50%, higher/lower than the previous values. For every change in the importance factors, the dose is re-optimized and the plan is then re-evaluated. The planning process proceeds in an iterative fashion, as shown in FIG. 1, until a desired solution is obtained. The local importance factors (i.e. sub-volume dependent parameters) could also be introduced in step I to affect the local dosimetric behavior in a non-uniform manner.

The introduction of the local importance factors or other similar local parameters makes it possible to identify the system parameters that are most responsible for the dosimetric behavior at a local level. It is this link that makes dose shaping more directly. The adjustment of the local importance factors can be performed sequentially or simultaneously for a few structures.

The regions or sub-volumes of interest could be graphically identified. For this purpose dose distribution layouts can be used to as guidance for geometrically selecting the regions or sub-volumes of interest where the dose(s) need to be modified by changing the local importance factors.

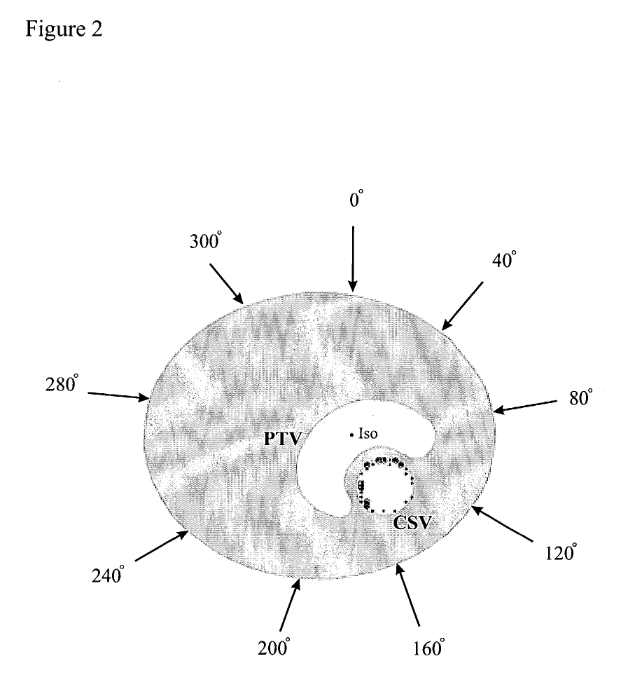

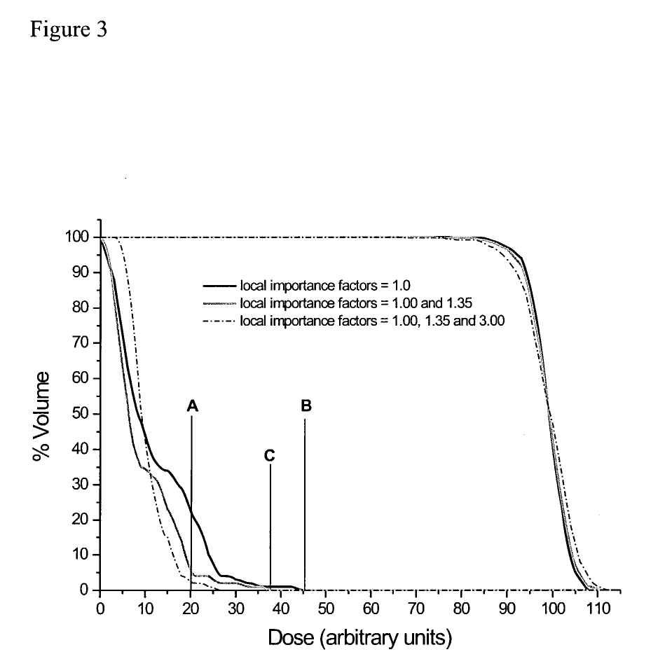

In one exemplary embodiment, the method of the present invention is shown for an elliptical phantom with a C-shaped tumor and an abutting circular critical structure (See FIGS. 2-3). The configuration of the C-shaped tumor case is shown in FIG. 2. Nine 6MV equispaced beams were used in the treatment (0°, 40°, 80°, 120°, 160°, 200°, 240°, 280°, and 320°—respecting the IEC convention). The prescribed dose to the PTV was set to 100 arbitrary dose units and 20 units were assigned as tolerance dose of the critical structure volume (CSV). Using the conventional inverse planning procedure, it was found that the values of the structure specific importance factors are r_{PTV}=0.8 and r_{CSV}=0.2. This set of importance factors provides a reasonable overall tradeoff between dose coverage of the tumor and the protection of the critical structure. The black lines in FIG. 3 show the tumor and critical structure DHVs for the plan optimized with this set of structure-specific importance factors. Assume that the clinical concern relates to the dose of the CSV, then one might want to lower the maximum dose and the fractional volume receiving dose in the interval AB shown in FIG. 3. This could be accomplished by first determining or identifying the responsible voxels by analyzing the dose distribution in the critical structure. These voxels represent ˜25% of the structure volume and are marked in FIG. 2 by plain dots. The distribution of the dots is along the periphery of the CSV's contour, with a larger density within the part proximal to the PTV. In a first attempt, the local importance factors for these voxels labeled by the plain dots were increased from 1.00 to 1.35, while the importance factors of the rest of the CSV voxels remained unchanged and fixed at unit value. Upon re-optimization of the system, the new DVHs are shown in gray lines in FIG. 3. The target coverage remains practically unchanged, but the CSV sparing is greatly improved. In particular, the maximum dose is decreased by almost 8 dose units as compared to the plan performed with only structure-specific importance factors. With the use of the local importance factors, the number of voxels that received a dose exceeding the tolerance level was greatly reduced. These voxels can now be found only at the boundary region with the PTV, as represented by open circles in FIG. 2. Further decrease of the fractional volume in the dose range A and C (see FIG. 3) could be sought in an attempt to improve the dose to the CSV. Therefore one could assign a new local importance value of 3.0 to the voxels labeled with open circles in FIG. 2 and then repeat the procedure as discussed supra. In this exemplary embodiment, the importance factors of the remaining voxels were kept at the same values that were used in the previous optimization (i.e., 1.35 for the voxels labeled by the plain dots and 1.0 for the voxels that are not labeled by circles or dots). The DVHs of the new plan corresponding to this distribution of the importance factors are shown as dotted lines in FIG. 3. The maximum dose of the CSV has dropped by 20 dose units as compared to the initial optimization result. The increased importance values for the CSV voxels lead to an increased dose inhomogeneity within the target. This is not surprising because of the trade-off nature of the problem. The important point here is that, when local importance factors are used, the trade-off is accentuated at a regional level and the control over the shapes of the final DVHs is greatly enhanced.

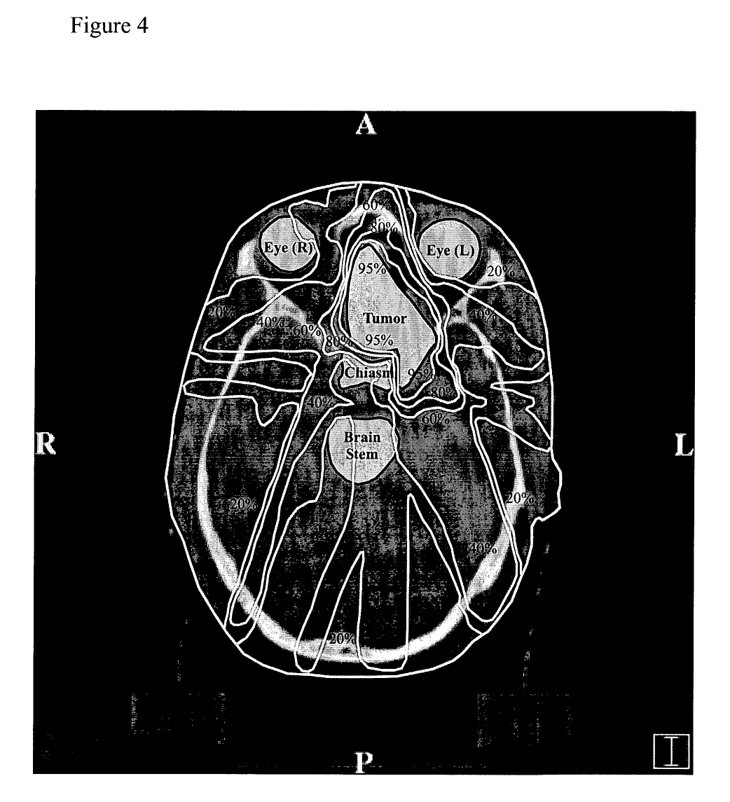

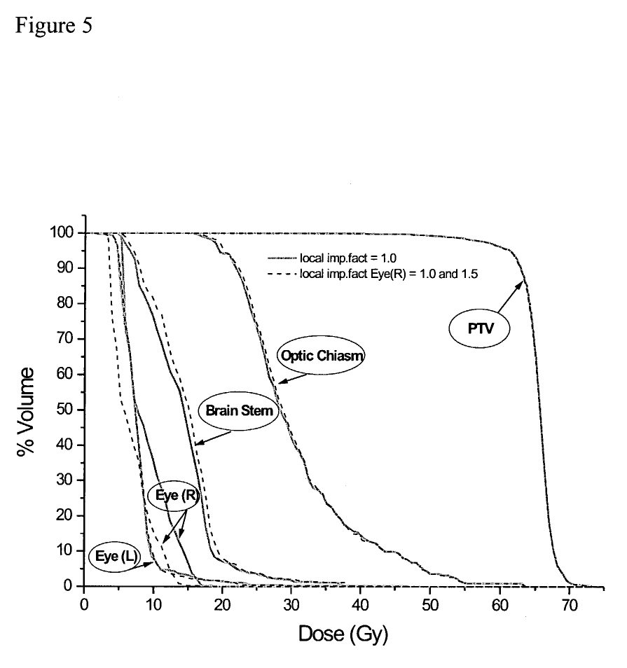

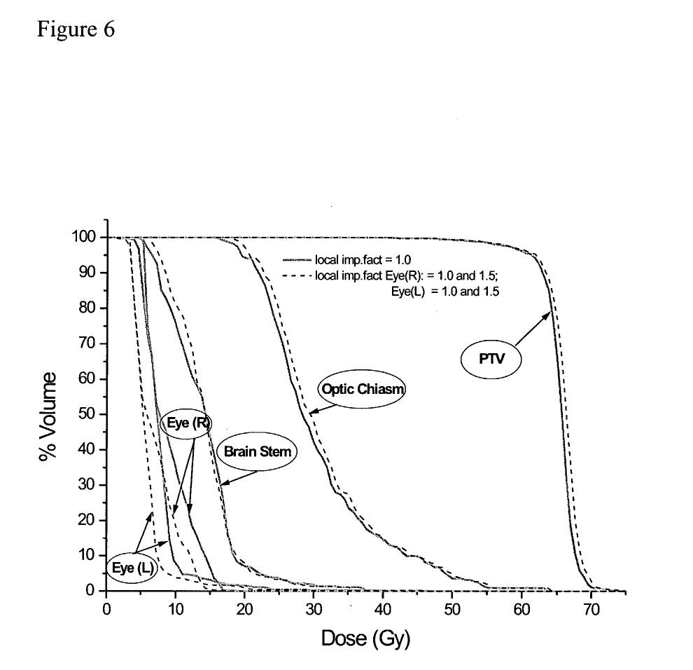

In another exemplary embodiment, the method of the present invention is shown for a nasopharinx tumor treatment plan in which several critical structures needs to be considered such as the eyeballs, optic chiasm and the brain stem. The prescription dose to the nasopharinx tumor was 60 Gy, and the tolerance doses were 10 Gy for the eyeballs, 35 Gy for the brain stem and 45Gy for the optic chiasm, respectively. Nine beams were placed at the following angular positions: 10°, 80°, 120°, 160°, 180°, 200°, 240°, 270° and 355°. The size of the pencil beam defined at the isocenter was 0.5 cm. An initial plan was obtained with the following set of structure-specific importance factors: 0.40 for the tumor, 0.32 for the right eye, 0.10 for the left eye, 0.04 for the brain stem 0.04 for the optic chiasm and 0.1 for the normal tissue, respectively. FIG. 4 shows the resulting isodose distribution in a transverse slice of the skull. In this case, it was found that the 95% isodose line covers acceptably well the PTV. The DVHs of the optimized plan are plotted with plain lines in FIG. 5. In a first instance, it might be desirable to lower the dose to the right eye. To lower the dose to the right eye, one could locate the voxels with a dose exceeding, for instance, the 10 Gy tolerance level and increase their importance from 1.0 to 1.5. The beam profile was re-optimized and the resulting DVHs are shown with dashed lines in FIG. 5. The results show no degradation of the target coverage and a significant reduction of the dose to the right eye accompanied by a reduction in the maximum dose by almost 5Gy. While the DVH curve for the other eye remains the same, an insignificant degradation is observed for the brain stem and optic chiasm. In an attempt to further increase the values of the local importance factors to 1.5 for those voxels receiving a dose higher than 10 Gy in both eyes. The dashed curves in FIG. 6 represent the corresponding DVHs of various structures after dose optimization. As in the previous case, the dose-volume characteristics of both eyes are improved significantly. Interestingly, the dose homogeneity in the PTV is also improved slightly.

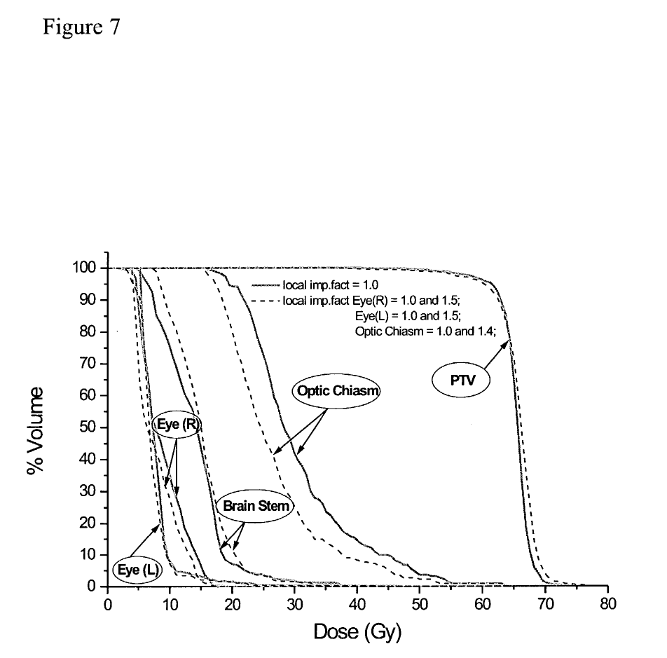

FIG. 6 shows that 15% of the optic chiasm receives a dose greater than 40 Gy. In another exemplary embodiment, this volume could be lowered and thereby the maximum optic chiasm dose be reduced. The voxels in the optic chiasm that could be considered as overdosed are identified and assigned with a new importance value of 1.4. The importance factor distributions in both eyes and other structures could be kept to the same as in the previous case. The DVHs corresponding to this new arrangement of the importance factors are shown in FIG. 7. While the optic chiasm DVH was significantly improved, the dose inhomogeneity within the tumor increased. In addition, the level of improvement in the eyes resulted from the last trial has worsen, even though it did not go back to the original plan shown as the plain curves in FIG. 7. This result suggests that the order in which the critical structures are considered into the dose-tuning process might play a role. If a critical structure is closely located to the target, the boundary region is usually in the overlap area of several beamlets coming from different beams. In general, the dose in this type of structures is more strongly correlated with that of other structures. It is also instructive to point out that the whole dose volume curve of the optic chiasm was improved as shown in FIG. 7 instead of only the dose bins above 40 Gy. This revealed the role of correlation between different voxels within the same structure, which is most pronounced for a structure like optic chiasm because of its small volume.

In yet another exemplary embodiment, the method of the present invention is shown for a prostate cancer treatment plan. In this example, the sensitive structures include the rectum, bladder and femoral heads. The IMRT treatment uses six co-planar beams with gantry angles of 0, 55, 135, 180, 225 and 305 degrees in IEC convention. Using the conventional inverse planning procedure a set of optimal structure specific importance factors are obtained and listed in Table 1, along with the relative prescription doses used for the optimization.

TABLE 1
Example of parameters used for obtaining the prostate conventional
optimized plan (OAR stands for Organ At Risk).

| | | Target prescription and |
|---|---|---|
| | Relative importance factors | OAR tolerance doses |
| GTV | 0.20 | 1.00 |
| Bladder | 0.05 | 0.60 |
| Rectum | 0.05 | 0.65 |
| Femural Head (R) | 0.05 | 0.45 |
| Femural Head (L) | 0.05 | 0.45 |
| Tissue | 0.60 | 0.60 |

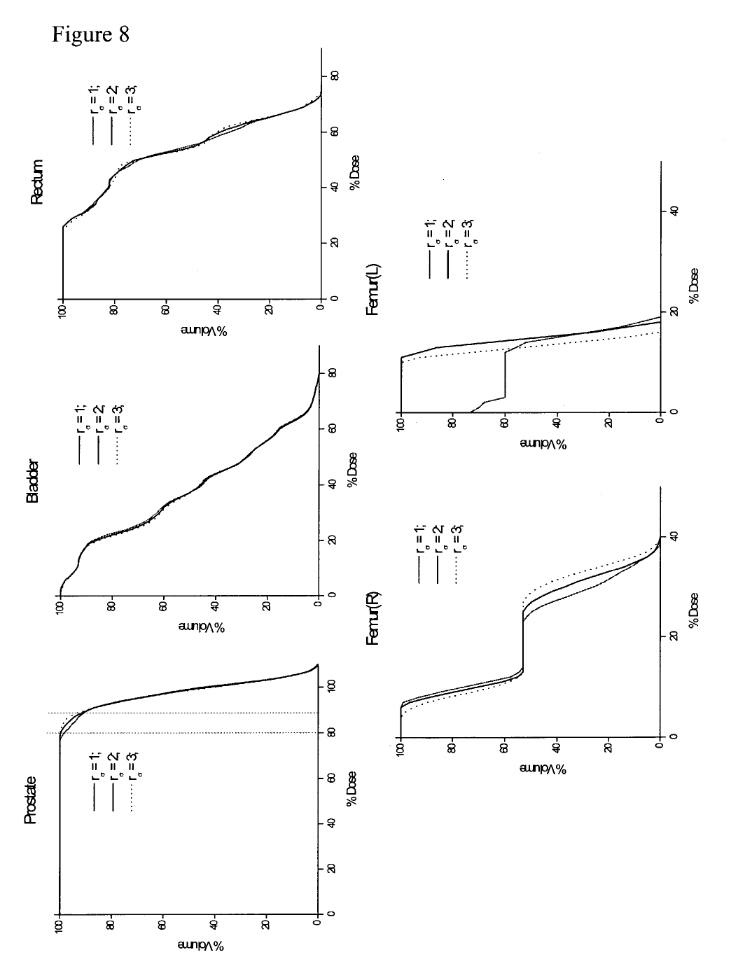

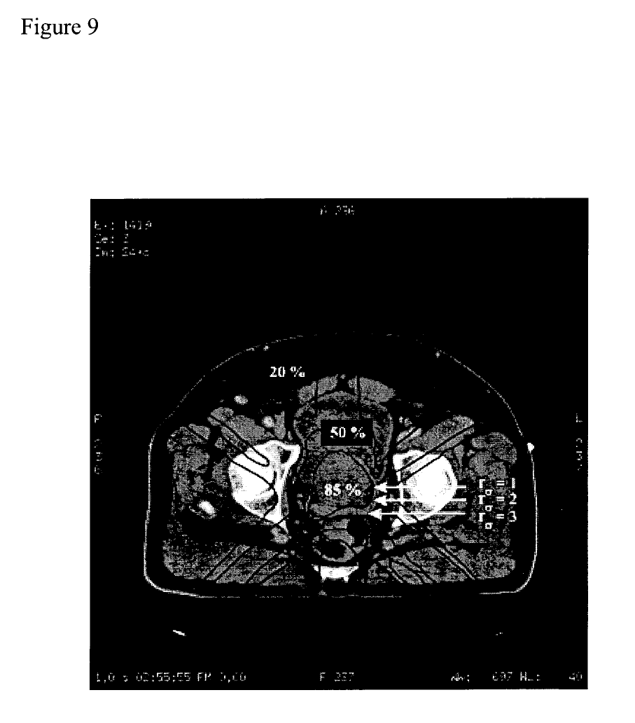

The DVHs of the structures involved in the conventional inverse plan are shown in FIGS. 8(a)-(e) in gray solid lines. Inspecting the target DVH shown in FIG. 8a, it was noticed that a fairly large fraction of the prostate volume receives a dose less than 88% of the prescription. Assuming that the clinical objective is to increase the fractional prostate volume receiving a dose less than 88% (shown between the two vertical lines in FIG. 8a), two successive fine-tunings could be performed. In the first attempt, based on the DVH data, the responsible voxels were identified and a higher importance, rₙ=2.0 was assigned to these voxels. In a second attempt the prostate coverage was further improved. The voxels that were under-dosed (below 88%) after the first trial were identified and the importance factor of these newly identified voxels further increased to 3.0. The results after re-optimization are shown as dotted lines. FIGS. 8(b)-(e) show the effect of increasing the local importance factors on the DVHs of the involved sensitive structures. As it can be obtained from FIG. 8, the local importance factors are able to fine-tune the target doses. For instance, the prostate volume covered by the 85% isodose curve was increased by 5% after the two trials. FIG. 9 shows the 85% isodose lines corresponding to the three optimized plans. The isodose line corresponding to the plan obtained with the largest voxel-based importance factors has the best target coverage and this is most distinct at the left posterior part of the prostate target.

The bladder and rectum suffered minor but practically insignificant changes when the local importance factors were increased. The differences in the femoral head doses might be important, especially in the left one, where approximately 40% more of its volume got irradiated as the prostate dose coverage was improved. Physically, this effect was produced by the intensity increase in a set of beamlets in the left anterior beam (gantry angle 55 degrees). The improvement in the dose to a structure is sometimes accompanied by the dosimetrically adverse effect(s) at other points in the same or different structures. The important point that one should note is that from the clinical point of view, some dose distributions are more acceptable than others and in one aspect it is the goal of the present invention to find the solution that improves the plan to the largest possible extent, but with a clinically insignificant or acceptable sacrifice. To achieve this, it is necessary to have a reasonable amount of controllability degree over the final dose distribution.

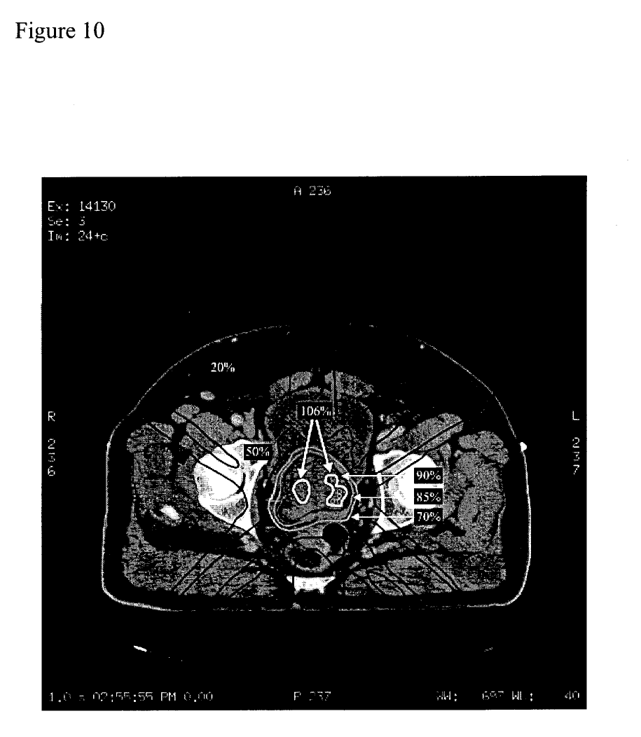

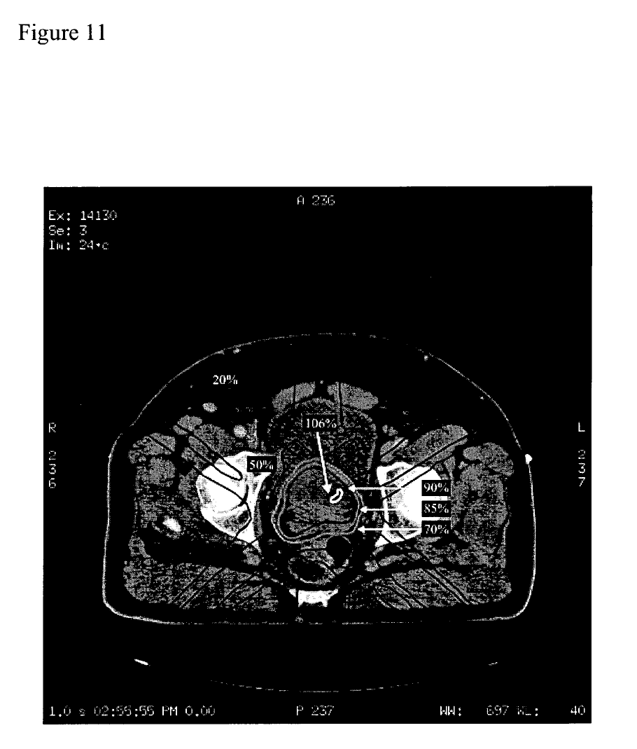

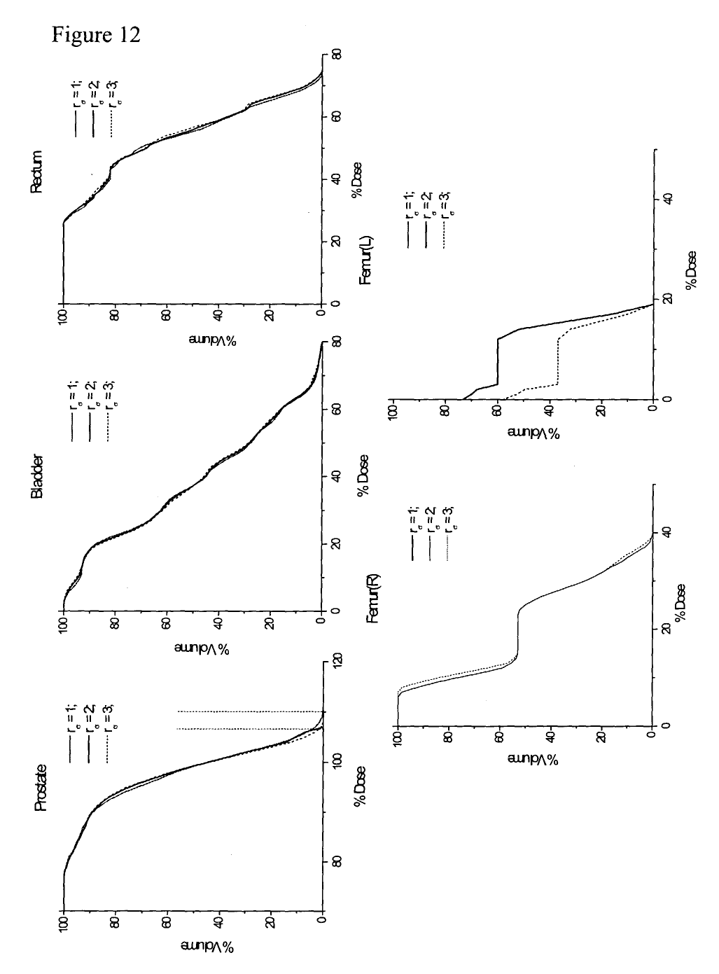

Another scenario that one could consider is the reduction of a tumor hot spot within the prostate target. Inspecting the target DVH shown in FIG. 8a, it can be seen that there is a small number of voxels in the prostate that receive a dose higher than 106%. This is more clearly shown in the dose layout shown in FIG. 10, where two tumor hot spots are found. It could be assumed that the clinical objective is now to reduce the doses to these two tumor hot spots, particularly to the one near the center of the prostate. For this purpose, one could graphically identify the tumor hot spots and then assign a higher importance (rₙ=2.0 in the first attempt, and rₙ=3.0 in the second attempt) to the corresponding voxels. FIG. 11 shows the isodose distribution after re-optimization. The tumor hot spot near the urethra disappeared and the size of the other hot spot was reduced significantly. This improvement is also evident in the DVH shown in FIG. 12a. The gray curves in FIGS. 12a-e correspond to the conventionally optimized plan (r_{σ}=1.0) while the plans obtained by introducing voxel-importance factors are shown with black solid lines (rₙ=2.0) and dotted lines (rₙ=3.0), respectively. As the value of rₙ increases, the role of the selected voxels becomes more important, forcing the system to satisfy the dosimetric requirements at the selected voxels. Similar to the precedent scenario, the DVHs of bladder and rectum remained practically unchanged after the dose shaping. The major difference occurred at the left femural head, when rₙ=3.0. As expected, to reduce the doses to the hot regions of the conventional plan, the intensity of the beamlets affecting both the femoral heads and the prostate (the tumor hot regions) became smaller. Accordingly, the dose to the intervening femoral head was reduced. This is opposite to the effect described supra, where the goal was to reduce the underdosage in the prostate. Nevertheless, the improvements in both cases were accomplished without violating the constraint of the left femoral head.

The present invention has now been described in accordance with several exemplary embodiments, which are intended to be illustrative in all aspects, rather than restrictive. It will be clear to one skilled in the art that the above embodiments may be altered in many ways without departing from the scope of the invention. Thus, the present invention is capable of many variations in detailed implementation, which may be derived from the description contained herein by a person of ordinary skill in the art. For example, the changes in local importance factors or sub-volume dependent parameter of different structures could be accomplished sequentially or simultaneously. The method was described in relation to IMRT but can also be applied for dose optimization in other radiation modalities, e.g., brachytherapy, stereotactive radio-surgery, gamma knife, modulated electron or proton therapy, cyber knife, etc. All such variations are considered to be within the scope and spirit of the present invention as defined by the following claims and their legal equivalents.

What is claimed is:

1. A method for determining an intensity modulated radiation treatment plan for a patient, comprising the step of assigning structurally non-uniform parameters to an objective function that is used to determine said intensity modulated radiation treatment plan for said patient.

2. The method as set forth in claim 1, wherein said non-uniform parameters are voxel-based parameters or sub-volume-based parameters that control the degree of penalty at said corresponding voxels or said corresponding sub-volumes.

3. A method for determining a radiation dose distribution in a target volume, wherein said target volume comprises one or more sub-volumes, comprising the steps of:
(a) assigning one or more sub-volume dependent parameters to develop a dosimetric behavior for said one or more sub-volumes;
(b) evaluate said dosimetric behavior of said one or more sub-volumes;
(c) changing said one or more sub-volume dependent parameters in a non-uniform manner to change said dosimetric behavior of said one or more sub-volumes; and
(d) optimizing said radiation dose distribution for said target volume with said changed one or more sub-volume dependent parameters.

4. The method as set forth in claim 3, wherein said one or more sub-volumes are one or more voxels.

5. The method as set forth in claim 3, wherein one or more sub-volumes comprises healthy tissue.

6. The method as set forth in claim 3, wherein one or more sub-volumes comprises unhealthy tissue.

7. The method as set forth in claim 3, wherein one or more sub-volumes comprises sensitive tissue.

8. The method as set forth in claim 3, wherein said step of evaluating said dosimetric behavior is evaluating a plan statistics graph, evaluating isodose layouts or evaluating dose-volume histograms.

9. The method as set forth in claim 3, further comprising the step of determining one or more non-uniform parameters controlling the degree of regional penalty for said target volume.

10. A program storage device accessible by a computer, tangibly embodying a program of instructions executable by said computer to perform method steps for determining a radiation dose distribution of a target volume, wherein said target volume comprises one or more sub-volumes, said method steps comprising:
(a) assigning one or more sub-volume dependent parameters to develop a dosimetric behavior for said one or more sub-volumes;
(b) evaluate said dosimetric behavior of said one or more sub-volumes;
(c) changing said one or more sub-volume dependent parameters in a non-uniform manner to change said dosimetric behavior of said one or more sub-volumes; and
(d) optimizing said radiation dose for said target volume with said changed one or more sub-volume dependent parameters.

11. The program storage device as set forth in claim 10, wherein said one or more sub-volumes are one or more voxels.

12. The program storage device as set forth in claim 10, wherein one or more sub-volumes comprises healthy tissue.

13. The program storage device as set forth in claim 10, wherein one or more sub-volumes comprises unhealthy tissue.

14. The program storage device as set forth in claim 10, wherein one or more sub-volumes comprises sensitive tissue.

15. The program storage device as set forth in claim 10, wherein said step of evaluating said dosimetric behavior is evaluating a plan statistics graph, evaluating isodose layouts or evaluating dose-volume histograms.

16. The program storage device as set forth in claim 10, further comprising the step of determining one or more non-uniform parameters controlling the degree of regional penalty for said target volume.
